(19) United States
(12) Patent Application Publication
(10) Pub. No.: US 2026/0216275 A1
(43) Pub. Date: Jul. 30, 2026

(54) FARFARAE FLOS AND ASTERIS RADIX ET RHIZOMA COUGH-RELIEVING GRANULE COMPOSITION AND PREPARATION METHOD THEREFOR

(71) Applicant: JIANGZHONG PHARMACEUTICAL CO., LTD., Nanchang, Jiangxi Province (CN)

(72) Inventors: Yanxia XIONG, Nanchang (CN); Wanwan KUANG, Nanchang (CN); Yangming WU, Nanchang (CN); Wenjun LIU, Nanchang (CN); Yiguang LI, Nanchang (CN); Ting WAN, Nanchang (CN); Jiawei HU, Nanchang (CN); Jinzhen XU, Nanchang (CN); Hanjing SUN, Nanchang (CN); Nana XIAO, Nanchang (CN)

(21) Appl. No.: 19/143,296

(22) PCT Filed: Feb. 7, 2024
(86) PCT No.: PCT/CN2024/076594

§ 371 Date: Jun. 25, 2025

(30) Foreign Application Priority Data
Dec. 30, 2022 (CN) 202211741691.1

(51) Int. Cl. A61K 36/9068; A61K 9/14; A61K 36/17; A61K 36/268; A61K 36/28; A61K 36/575; A61K 36/8888; A61K 47/69; C12N 9/42

(52) U.S. Cl. CPC A61K 36/9068; A61K 9/145; A61K 9/146; A61K 36/17; A61K 36/268; A61K 36/28; A61K 36/575; A61K 36/8888; A61K 47/6951; C12N 9/2437; A61K 2236/19; A61K 2236/331; A61K 2236/37; A61K 2236/39; A61K 2236/53; C12Y 302/01004

(57) ABSTRACT

A Farfarae flos and Asteris radix et rhizoma cough-relieving granule composition and a preparation method therefor. The composition comprises the following components: an inclusion complex, an extract powder and a filler comprising lactose and dextrin. The preparation method therefor comprises: extracting Zingiberis rhizoma recens, Asari radix et rhizoma and Magnoliae flos to obtain an volatile oil and medicine residue 1; then performing enzymolysis on Ephedrae herba to obtain an enzymolysis solution and medicine residue 2; decocting Asteris radix et rhizoma, Farfarae flos, Pinelliae rhizoma praeparatum, Xanthii fructus, the medicine residue 1 and the medicine residue 2 with water, combining same with the enzymolysis solution, and then drying same to obtain the extract powder; then performing the inclusion of the volatile oil to obtain the inclusion complex; and finally, mixing the inclusion complex, the extract powder and the filler for granulation to obtain the Farfarae flos and Asteris radix et rhizoma cough-relieving granule composition.

### TECHNICAL FIELD

The present disclosure belongs to the field of traditional Chinese medicine, and in particular relates to a composition of Dongwan cough-relieving granules and a preparation method thereof.

### BACKGROUND OF THE INVENTION

The formula of Dongwan cough-relieving granule is an empirical prescription established by Professor Hong Guang-xiang on the basis of ancient cough relieving experience, drawing the prescription characteristics of Shegan Ephedra Decoction, etc. and combining the etiology and pathogenesis of wind-cold cough and his own clinical experience. The formula of Dongwan cough-relieving granule can be referred to the patent CN1103224C. For acute bronchitis (exogenous wind-cold syndrome), the indications are prominent and clear. By dispersing the lung, the pathogen of exogenous wind-cold can be quickly expelled from the surface, and the cough can be cured due to the potential. It has the effects of dispersing the lung, relieving cough, relieving cough and calming asthma, etc. and is an important medicine for exogenous cough.

Granule dosage has the advantages of large drug loading, quick-acting and convenient carrying, but traditional granules mostly adopt wet granulation technology, wet granulation has the advantages of simple equipment, low investment, low energy consumption, high production efficiency, etc. But at the same time, there are many defects, such as excessive excipient dosage, low mechanization degree, many operating procedures, susceptibility to microbial contamination, etc. And the excipients used in wet granulation process are usually limited to starch, sugar powder and dextrin, the excipient dosage is determined depending on empirical judgment on extract proportion, extract consistency, etc., which compromises the formulation quality control. And when sucrose and dextrin are used as excipients, the amount of excipients is large, causing the particles to easily absorb moisture, and obesity, diabetes patients are not suitable for taking them.

In the original formulation process of Dongwan cough-relieving granule, steam distillation was used to extract volatile oil from three medicinal materials including ginger, Herba Asari and Magnolia Flower. The yield of volatile oil was generally low in production, and the volatile oil directly sprayed in granules is volatile easily, which affects the stability and curative effect of formulation. Ephedra and the other five herbs were decocted in water, resulting in a low extraction rate of ephedrine. After refining, the extract powder obtained by spray drying is prone to moisture absorption and sticky, which leads to difficulty in granulating. The original technology adopts sucrose as filling agent, causing obesity, diabetes patients not suitable to take, etc.

Therefore, the present disclosure conducts research on the molding process of Dongwan cough-relieving granule, aiming to solve the problems in the prior art, such as low yield of volatile oil and direct spraying of volatile oil on granules affecting formulation stability, low extraction rate of active ingredients, easy prone to moisture absorption of extract powder, difficulty in granulation, and unsuitable for obesity and diabetes patients.

### SUMMARY OF THE INVENTION

In view of the deficiencies of the prior art, the present disclosure provides a Dongwan cough-relieving granule composition and a preparation method thereof. The process of the present disclosure is simple and stable, and the prepared granules have high transfer rate of active ingredients, precise therapeutic effect, and stable quality.

In order to achieve the object of the present disclosure, the following technical solutions are adopted:

A composition of Dongwan cough-relieving granules, wherein the raw materials of the composition comprise the following components: an inclusion complex, extract powder and a filler; and the filler includes lactose and dextrin.

Preferably, the raw materials of the composition comprise the following components in parts by weight: 5-15 parts of the inclusion complex, 30-50 parts of the extract powder and 50-60 parts of the filler.

Preferably, the mass ratio between lactose and dextrin is 4-6:1.

Preferably, the inclusion complex is formed by inclusion of the volatile oil extracted from all the medicinal materials of Dongwan granules within β-cyclodextrin; and the mass ratio of β-cyclodextrin to the volatile oil is 6-10:1.

Preferably, the inclusion is performed at a temperature of 45-55° C. for 45-75 min.

Another object of the present disclosure is to provide a preparation method for preparing the Composition of Dongwan cough-relieving granules according to any one of claims 1 to 4, characterized in comprising the following steps:
(1) extracting ginger, Herba Asari and Magnolia Flower to obtain a volatile oil and a medicinal residue 1;
(2) enzymatically hydrolyzing ephedra to obtain an enzymolysis solution and a medicinal residue 2;
(3) decocting Tatarian Aster Root, Tussilago Flower, Rhizoma Pinelliae Praeparatum, Xanthium Fruit, medicinal residue 1 and medicinal residue 2 in water, combining the decoction with the enzymolysis solution, filtering, and drying to obtain an extract powder;
(4) encapsulating the volatile oil to obtain an inclusion complex; and
(5) mixing the inclusion complex, the extract powder, and a filler, and granulating the mixture to obtain a composition of Dongwan cough-relieving granules.

Preferably, the enzyme used in the enzymatic hydrolysis in step (2) is cellulase, and the mass ratio of the ephedra to the enzyme is 100:0.3-1.2.

Preferably, the enzymatic hydrolyzing in step (2) is performed at a temperature of 45-65° C. and a pH of 4.0-6.5 for a duration of 1-4 h.

Preferably, the extracting in step (1) is supercritical CO₂ extraction, at an extraction pressure of 15-25 MPa, an extraction temperature of 30-50° C. for a duration of 1.5-2.5 h.

Preferably, the granulating in step (5) is one-step granulation, under the process conditions of a spray frequency of 10-15 Hz, an atomization pressure of 0.12-0.16 MPa, a material temperature of 40-55° C., an inlet air temperature of 70-90° C., and an exhaust air temperature of 30-50° C.

Compared with the prior art, the beneficial effects of the present disclosure are as follow:
(1) that disclosure selects and uses the lactose and the dextrin as filler, which can effectively reduce the hygroscopicity of the granule and improve the stability of Dongwan cough-relieving granules; expands the applicable patient population; can addressing issues in the original process such as difficult granulation and unsuitability for obese/diabetic patients.
(2) The present disclosure uses a specific process to encapsulate the volatile oil, which can cover the bad smell of the volatile oil well and improve the stability of the formulation.
(3) In the present disclosure, the flavoring agent is also screened, and the aspartame is selected as the flavoring agent, which can well mask the bitter taste of the Dongwan cough-relieving granules, is convenient to take, and has good patient compliance.
(4) The present disclosure selects a specific process and parameters, and, the prepared granules have good melting property, high granule qualification rate and high production efficiency, compared with other processes.
(5) Three-month accelerated test and long-term test were conducted on this product with proposed marketing package sample (double aluminum). The results show that: this product is stable for 3 months.

Pharmacodynamic and acute toxicological tests confirmed that the Dongwan cough-relieving granules of the present novel technical process have excellent pharmacodynamic indexes, a little toxicity by oral administration, safety use, and effects such as cough relieving, phlegm relieving, asthma smoothing.

### DETAILED DESCRIPTION OF THE INVENTION

The present disclosure will be further described below with reference to specific embodiments.

### Example 1

(1) adding 6 times the amount of water to ginger, Herba Asari and Magnolia Flower, and extracting by supercritical CO₂, wherein the extraction pressure is 20 MPa, the extraction temperature is 30° C. and the extraction duration is 2 h, to obtain volatile oil and drug residue 1;
(2) enzymatically hydrolyzing ephedra with cellulase, wherein the temperature of the enzymatic hydrolysis is 50° C., the pH of the enzymatic hydrolysis is 5.0, the duration of the enzymatic hydrolysis is 1 h, and the mass ratio of the ephedra to the enzyme is 100:0.5; and the enzymolysis solution and drug residue 2 were obtained;
(3) decocting Tatarian Aster Root, Tussilago Flower, Rhizoma Pinelliae Praeparatum, Xanthium Fruit, medicinal residue 1 and medicinal residue 2 in water twice, with the first duration of 1.5 h and the second duration of 1.0 h, then the decoction liquids were combined, the decoction liquids were combined with the enzymolysis solution, and the resultant were centrifuged under freezing condition at 5000 r/min and 15° C. for about 30 min and filtered to obtain a clear liquid, which was concentrated and dried at 60° C. and −0.085 to 0.01 Mpa, to obtain extract powder by drying;
(4) encapsulating the volatile oil in a manner that β-cyclodextrin was accurately weighed, placed in an Erlenmeyer flask with a stopper, distilled water was added therein, the resultant was heated in boiling water bath to dissolve, and a clear solution was prepared. The system was reduced to the specified temperature of 50° C., and kept at constant temperature as the inclusion temperature, stirred on a magnetic stirrer at constant temperature. A pipette is used to measure the volatile oil, the volatile oil was slowly dropped into the β-cyclodextrin solution maintained at a certain temperature at a stirring speed of 25 r/min, a stopper was used to cover the inlet. The system was continuously kept at constant temperature for 1 hour, then placed in a refrigerator at 4° C. for 24 hours. The system was subjected to suction filter, drain, and then the inclusion complex was washed with 10 ml of absolute ethanol respectively, the filter residue was collected, and dried in an oven at 40° C. to obtain the inclusion complex; the mass ratio of β-cyclodextrin to volatile oil was 8:1.
(5) Taking the extract powder 50 g, total 60 g of lactose and dextrin (5:1), the inclusion complex 10 g, mixing them well as base material. 3% of prescription amount pure water was added as adhesive, one-step granulation was performed. The conditions were set as the spraying frequency to 12 Hz, the atomization pressure to 0.14 MPa, the air inlet temperature to 70° C., and the air exhaust temperature to 30° C. The granules were dried and sized, and collected to obtain the composition of Dongwan cough-relieving granules.

### Example 2

(1) adding 8 times the amount of water to ginger, Herba Asari and Magnolia Flower, and extracting by supercritical CO₂, wherein the extraction pressure is 15 MPa, the extraction temperature is 50° C. and the extraction duration is 1.5 h, to obtain volatile oil and drug residue 1;
(2) enzymatically hydrolyzing ephedra with cellulase, wherein the temperature of the enzymatic hydrolysis is 45° C., the pH of the enzymatic hydrolysis is 6.0, the duration of the enzymatic hydrolysis is 3 h, and the mass ratio of the ephedra to the enzyme is 100:0.3; and the enzymolysis solution and drug residue 2 were obtained;
(3) decocting Tatarian Aster Root, Tussilago Flower, Rhizoma Pinelliae Praeparatum, Xanthium Fruit, medicinal residue 1 and medicinal residue 2 in water twice, with the first duration of 1.5 h and the second duration of 1.0 h, then the decoction liquids were combined, the decoction liquids were combined with the enzymolysis solution, and the resultant were centrifuged under freezing condition at 5000 r/min and 15° C. for about 30 min and filtered to obtain a clear liquid, which was concentrated and dried at 60° C. and −0.085 to 0.01 Mpa, to obtain extract powder by drying;
(4) encapsulating the volatile oil in a manner that β-cyclodextrin was accurately weighed, placed in an Erlenmeyer flask with a stopper, distilled water was added therein, the resultant was heated in boiling water bath to dissolve, and a clear solution was prepared. The system was reduced to the specified temperature of 45° C., and kept at constant temperature as the inclusion temperature, stirred on a magnetic stirrer at constant temperature. A pipette is used to measure the volatile oil, the volatile oil was slowly dropped into the β-cyclodextrin solution maintained at a certain temperature at a stirring speed of 25 r/min, a stopper was used to cover the inlet. The system was continuously kept at constant temperature for 1 hour, then placed in a refrigerator at 4° C. for 24 hours. The system was subjected to suction filter, drain, and then the inclusion complex was washed with 10 ml of absolute ethanol respectively, the filter residue was collected, and dried in an oven at 40° C. to obtain the inclusion complex; the mass ratio of β-cyclodextrin to volatile oil was 6:1.
(5) Taking the extract powder 50 g, total 50 g of lactose and dextrin (4:1), the inclusion complex 10 g, mixing them well as base material. 3% of prescription amount pure water was added as adhesive, one-step granulation was performed. The conditions were set as the spraying frequency to 10 Hz, the atomization pressure to 0.16 MPa, the air inlet temperature to 80° C., and the air exhaust temperature to 40° C. The granules were dried and sized, and collected to obtain the composition of Dongwan cough-relieving granules.

### Example 3

(1) adding 10 times the amount of water to ginger, Herba Asari and Magnolia Flower, and extracting by supercritical CO₂, wherein the extraction pressure is 20 MPa, the extraction temperature is 30° C. and the extraction duration is 2 h, to obtain volatile oil and drug residue 1;
(2) enzymatically hydrolyzing ephedra with cellulase, wherein the temperature of the enzymatic hydrolysis is 50° C., the pH of the enzymatic hydrolysis is 4.0, the duration of the enzymatic hydrolysis is 1 h, and the mass ratio of the ephedra to the enzyme is 100:1; and the enzymolysis solution and drug residue 2 were obtained;
(3) decocting Tatarian Aster Root, Tussilago Flower, Rhizoma Pinelliae Praeparatum, Xanthium Fruit, medicinal residue 1 and medicinal residue 2 in water twice, with the first duration of 1.5 h and the second duration of 1.0 h, then the decoction liquids were combined, the decoction liquids were combined with the enzymolysis solution, and the resultant were centrifuged under freezing condition at 5000 r/min and 15° C. for about 30 min and filtered to obtain a clear liquid, which was concentrated and dried at 60° C. and −0.085 to 0.01 Mpa, to obtain extract powder by drying;
(4) encapsulating the volatile oil in a manner that β-cyclodextrin was accurately weighed, placed in an Erlenmeyer flask with a stopper, distilled water was added therein, the resultant was heated in boiling water bath to dissolve, and a clear solution was prepared. The system was reduced to the specified temperature of 55° C., and kept at constant temperature as the inclusion temperature, stirred on a magnetic stirrer at constant temperature. A pipette is used to measure the volatile oil, the volatile oil was slowly dropped into the β-cyclodextrin solution maintained at a certain temperature at a stirring speed of 25 r/min, a stopper was used to cover the inlet. The system was continuously kept at constant temperature for 1 hour, then placed in a refrigerator at 4° C. for 24 hours. The system was subjected to suction filter, drain, and then the inclusion complex was washed with 10 ml of absolute ethanol respectively, the filter residue was collected, and dried in an oven at 40° C. to obtain the inclusion complex; the mass ratio of β-cyclodextrin to volatile oil was 9:1.
(5) Taking the extract powder 50 g, total 60 g of lactose and dextrin (5:1), the inclusion complex 10 g, mixing them well as base material. 3% of prescription amount pure water was added as adhesive, one-step granulation was performed. The conditions were set as the spraying frequency to 15 Hz, the atomization pressure to 0.12 MPa, the air inlet temperature to 90° C., and the air exhaust temperature to 50° C. The granules were dried and sized, and collected to obtain the composition of Dongwan cough-relieving granules.

### Comparative Example 1

This comparative example is same as Example 1 except that the inclusion temperature is 40° C.

### Comparative Example 2

This comparative example is same as Example 1 except that the inclusion temperature is 60° C.

### Comparative Example 3

In this comparative example, the ratio of β-cyclodextrin to volatile oil is 4:1.

### Comparative Example 4

In this comparative example, the ratio of β-cyclodextrin to volatile oil is 10:1.

### Comparative Example 5

This comparative example is same as Example 1 except that different filler was used, see Table 1 for details.

TABLE 1

| Name of | | | | | | | | | |
|---|---|---|---|---|---|---|---|---|---|
| raw and | | | | | | | | | |
| auxiliary | Sample No. | | | | | | | | |
| materials | 1 | 2 | 3 | 4 | 5 | 6 | 7 | 8 | 9 |
| Extract | 40 | 40 | 40 | 40 | 40 | 40 | 40 | 40 | 40 |
| Powder | | | | | | | | | |
| (g) | | | | | | | | | |
| Lactose | 60 | / | / | / | / | 30 | 45 | 15 | 10 |
| (g) | | | | | | | | | |
| Mannitol | / | 60 | / | / | / | / | / | / | / |
| (g) | | | | | | | | | |
| Dextrin | / | / | 60 | / | / | 30 | 15 | 45 | 50 |
| (g) | | | | | | | | | |
| Sorbitol | / | / | / | 60 | / | / | / | / | / |
| (g) | | | | | | | | | |
| Soluble | / | / | / | / | 60 | / | / | / | / |
| starch (g) | | | | | | | | | |
| inclusion | 10 | 10 | 10 | 10 | 10 | 10 | 10 | 10 | 10 |
| complex | | | | | | | | | |

### Comparative Example 6

This comparative example adopted a dry granulation process, and the process parameters included a roller pressure 6.0 MPa, a roller rotation speed 12 r/min, and a granule rotation speed 35 r/min.

### Comparative Example 7

The process parameters of the one-step granulation in this comparative example were set as the spray frequency to 12 Hz, the atomization pressure to 0.18 MPa, the inlet air temperature to 95° C., and the exhaust air temperature to 55° C.

### Sample Determination

(1) The inclusion complex samples of Examples 1 to 3 and Comparative Examples 1 to 4 were measured, weighed, and the content of volatile oil in the inclusion complex was determined according to method according to Pharmacopoeia 2020 edition. Volatile oil yield and inclusion complex yield were calculated separately, and evaluated comprehensively. The comprehensive evaluation method is: the total score value is 100 points, wherein the inclusion complex yield rate accounts for 30 points, and the volatile oil inclusion rate accounts for 70 points. The results are as follows in Table 2:

| Temperature | | | | Comp. | Comp. | Comp. | Comp. |
|---|---|---|---|---|---|---|---|
| (° C.) | Ex. 1 | Ex. 2 | Ex. 3 | Ex. 1 | Ex. 2 | Ex. 3 | Ex. 4 |
| Volatile oil | 96.67 | 91.11 | 93.33 | 61.10 | 66.70 | 72.51 | 91.10 |
| inclusion | | | | | | | |
| rate (%) | | | | | | | |
| Inclusion | 81.66 | 79.80 | 82.31 | 72.88 | 59.32 | 75.08 | 67.04 |
| complex | | | | | | | |
| yield (%) | | | | | | | |
| comprehen- | 91.14 | 90.06 | 89.04 | 64.63 | 64.49 | 73.28 | 83.88 |
| sive score | | | | | | | |

Analysis of Results: when inclusion temperature is 45-50° C., inclusion rate and yield of volatile oil are high, comprehensive score is highest. Too high or too low temperature is not favor to inclusion of volatile oil.

(2) Comprehensively score on the granules prepared in Examples 1 to 3 and Comparative Examples 5 to 7. The results are shown in Tables 3 to 4:

Moisture Absorption Measurement is calculated according to the following formula:

$$Moisture Absorption Rate ( \% ) = \frac{\begin{matrix}Granules Weight after Moisture Absorption - \\ Granules Weight before Moisture Absorption\end{matrix}}{Granules Weight before Moisture Absorption} \times 100 \%$$

The particle size qualification rate was determined according to the double sieving method Appendix XIB of the Chinese Pharmacopoeia 2010 edition. 30 grams of particles was weighted under each process condition, to an exact weight, placed in a specified drug sieve. The granules were sieved horizontally by moving the sieve back and forth while gently tapping for 3 minutes. The particles that cannot pass through the No. 1 sieve and can pass through the No. 5 sieve were weighed, and the particle qualification rate was calculated.

$$Particle Qualification Rate = \frac{\begin{matrix}Weight of Qualified Particles \\ Between No . 1 Sieve and No . 5 Sieve\end{matrix}}{Weight of Dry Extract Powder + Weight of Excipients} \times 100 \%$$

TABLE 3
Comprehensive Scoring Table of Particles

| | 72 h moisture | | Particle size | | |
|---|---|---|---|---|---|
| Prescription | absorption rate | score | qualified rate | score | total |
| No. | (%) | A | (%) | B | score |
| Example 1 | 10.83 | 47.29 | 90.16 | 51.11 | 98.40 |
| Example 2 | 11.02 | 45.72 | 88.91 | 47.46 | 93.18 |
| Example 3 | 11.39 | 42.68 | 91.05 | 53.71 | 96.39 |
| Comparative | 15.86 | 5.92 | 80.23 | 22.09 | 28.01 |
| Example 6 | | | | | |
| Comparative | 12.58 | 32.89 | 85.78 | 38.31 | 71.20 |
| Example 7 | | | | | |

$$Score A = ( 1 - \frac{moisture absorption rate - 10.5}{14.58 - 8.50} ) \times 50$$

$$Score B = \frac{Particle size qualified rate - 72.67}{89.78 - 72.67} \times 50$$

TABLE 4
Comprehensive Scoring Table of Particles
of Comparative Example 5

| | 72 h moisture | | Particle size | | |
|---|---|---|---|---|---|
| Sample | absorption rate | score | qualified rate | score | total |
| No. | (%) | A | (%) | B | score |
| 1 | 10.50 | 50.00 | 85.30 | 36.91 | 86.91 |
| 2 | 12.32 | 27.70 | 79.29 | 19.35 | 47.05 |
| 3 | 11.73 | 34.93 | 89.78 | 50.00 | 84.93 |
| 4 | 13.34 | 15.20 | 72.67 | 0 | 15.20 |
| 5 | 14.58 | 0 | 78.54 | 17.74 | 17.74 |
| 6 | 11.94 | 16.92 | 91.05 | 50.00 | 24.90 |
| 7 | 11.02 | 40.15 | 88.91 | 29.69 | 64.06 |
| 8 | 12.36 | 6.31 | 85.78 | 0 | 6.31 |
| 9 | 12.61 | 0 | 88.04 | 21.44 | 21.44 |

Analysis of Results: the one-step granulation process and its parameters, and different excipients have obvious effects on the properties of the prepared granules. The inventor found that after lactose and dextrin are compounded at a ratio of 6-9:1, it is better in comprehensive evaluation aspects such as moisture absorption rate at 72 hours and particle size qualified rate, the moldability is good and moisture resistance is strong. By using mixtures of lactose and dextrin in other proportions, and excipients such as lactose, mannitol, sorbitol, dextrin or soluble starch, the quality of the granules is inferior to that of the formula of the present disclosure.

### (3) Examination of Appearance and Dissolution Rate of Granules

The granules prepared in Examples 1 to 3 and Comparative Examples 5 to 7 were comprehensively scored. The results are shown in Table 5:

TABLE 5

| | | Melting |
|---|---|---|
| Sample | Appearance | time (s) |
| Example 1 | Round shape, smoother | 54 |
| Example 2 | surface | 54 |
| Example 3 | | 53 |
| Comparative | Irregular shape, rougher | 188 |
| Example 6 | surface and color difference | |
| Comparative | rougher surface | 95 |
| Example 7 | | |

TABLE 6

| Sample | | Melting |
|---|---|---|
| No. | Appearance | time (s) |
| 1 | Round shape, smoother | 40 |
| 2 | surface, but more fine | 43 |
| 3 | powder | 43 |
| 4 | | 48 |
| 5 | | 44 |
| 6 | Round shape, smoother surface | 53 |
| 7 | rounding smooth | 50 |
| 8 | smoother surface | 70 |
| 9 | smoother surface | 75 |

Analysis of Results: different granulation techniques have significant impacts on the dissolution properties of granules. The one-step granulation method of this disclosure, coupled with its superior granulation parameters and appropriate compatibility of excipients, results in granules with rounder shapes, smoother surfaces, and better dissolution performance.

The above detailed description is a specific description of one of the possible embodiments of the present disclosure, and the embodiments are not intended to limit the patent scope of the present disclosure. Any equivalent implementation or modification without departing from the present disclosure should be included in the scope of the technical solution of the present disclosure.

1. A composition of Dongwan cough-relieving granules, characterized in that raw materials of the composition comprise the following components: an inclusion complex, extract powder, and a filler, wherein and the filler includes lactose and dextrin.

2. The composition according to claim 1, characterized in that the raw materials of the composition comprise the following components in parts by weight: 5-15 parts of the inclusion complex, 30-50 parts of the extract powder, and 50-60 parts of the filler.

3. Composition according to claim 1, characterized in that the mass ratio between lactose and dextrin is 4-6:1.

4. The composition according to claim 1, characterized in that the inclusion complex is formed by inclusion of volatile oil extracted from all medicinal materials of the Dongwan cough-relieving granules within β-cyclodextrin, wherein the mass ratio of β-cyclodextrin to the volatile oil is 6-9:1.

5. The composition according to claim 4, characterized in that the inclusion is performed at a temperature of 45-55° C. for 45-75 minutes.

6. A method for preparing the composition of Dongwan cough-relieving granules according to claim 1, comprising the following steps:
(1) extracting ginger, Herba Asari, and Magnolia Flower to obtain a volatile oil and a medicinal residue 1;
(2) enzymatically hydrolyzing ephedra to obtain an enzymolysis solution and a medicinal residue 2;
(3) decocting Tatarian Aster Root, Tussilago Flower, Rhizoma Pinelliae Praeparatum, Xanthium Fruit, medicinal residue 1, and medicinal residue 2 in water, combining the decoction with the enzymolysis solution, filtering, and drying to obtain an extract powder;
(4) encapsulating the volatile oil to obtain an inclusion complex; and
(5) mixing the inclusion complex, the extract powder, and a filler, and granulating the mixture to obtain a composition of Dongwan cough-relieving granules.

7. The method according to claim 6, characterized in that enzymatically hydrolyzing ephedra in step (2) is performed with a cellulase enzyme, and the mass ratio of the ephedra to the cellulase enzyme is 100:0.3-1.2.

8. The method according to claim 6, characterized in that the enzymatic hydrolyzing in step (2) is performed at a temperature of 45-65° C. and a pH of 4.0-6.5 for a duration of 1-4 hours.

9. The method according to claim 6, characterized in that; the extracting in step (1) is supercritical CO₂ extraction, at an extraction pressure of 15-25 MPa and an extraction temperature of 30-50° C. for a duration of 1.5-2.5 hours.

10. The method according to claim 6, characterized in that; the granulating in step (5) is one-step granulation, under the process conditions of a spray frequency of 10-15 Hz, an atomization pressure of 0.12-0.16 MPa, a material temperature of 40-55° C., an inlet air temperature of 70-90° C., and an exhaust air temperature of 30-50° C.
